(19) United States
(12) Patent Application Publication
(10) Pub. No.: US 2005/0059708 A1
(43) Pub. Date: Mar. 17, 2005

(54) Processes for making thiazolidinedione derivatives and compounds thereof

(76) Inventors: Karel Pospisilik, Brno (CZ); Frantisek Picha, Brno (CZ); Jie Zhu, Nijmegen (NL)

Correspondence Address: MARK R. BUSCHER, P.O. BOX 161, CATHARPIN, VA, 20143, US

(21) Appl. No.: 10/842,635

(22) Filed: May 11, 2004

Related U.S. Application Data
(60) Provisional application No. 60/469,837, filed on May 13, 2003.

(51) Int. Cl. C07D 213/72; C07D 213/55; A61K 31/44

(52) U.S. Cl. 514/352; 514/357; 546/304; 546/335

(57) ABSTRACT

A compound of the formula: 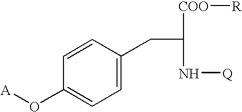
wherein A represents a ring group connected to the oxygen atom by a C₁ to C₆ hydrocarbon chain, R is hydrogen or a C₁-C₄ alkyl, and Q is hydrogen, or an amine protecting group such as acetyl, trifluoroacetyl, benzoyl, benzyl, or trityl, is useful in making thiazolidinedione derivatives 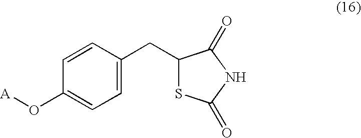 such as pioglitazone, rosiglitazone and troglitazone.

This application claims the benefit of priority under 35 U.S.C. § 119(e) from U.S. Provisional Patent Application Ser. No. 60/469,837, filed May 13, 2003, the entire contents of which are incorporated herein by reference.

### BACKGROUND OF THE INVENTION

The present invention relates to processes of manufacturing thiazolidinedione derivatives such as pioglitazone and to compounds useful in the processes.

Many thiazolidinedione derivatives or “glitazones” are known to exhibit hypoglycemic activity and/or blood lipid lowering activity and have been proposed for use in treating, inter alia, diabetes. Some of the more well known and/or studied glitazones include pioglitazone, troglitazone, and rosiglitazone. Pioglitazone, chemically 5-[[4-[2-(5-ethyl-2-pyridinyl)-ethoxy]phenyl]methyl]-2,4- thiazolidinedione of formula (1) 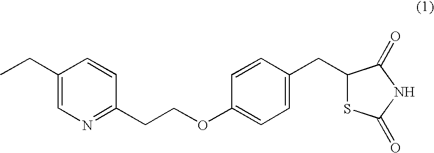
is a commercially approved antidiabetic agent. Pharmaceutical compositions comprising pioglitazone, as the hydrochloride salt, are marketed under the brand name ACTOS® (Takeda Chemical Ind.) for treatment of type II diabetes.

Pioglitazone and its hydrochloride have been disclosed in EP 193256 and corresponding U.S. Pat. No. 4,687,777. In these patents, the glitazone, such as pioglitazone, can be formed by cyclizing an alpha-bromo acid ester (2) with thiourea. The resulting imino-thiazolidinone (3) is then hydrolyzed to make the corresponding glitazone. For pioglitazone, the reaction can be represented as follows:

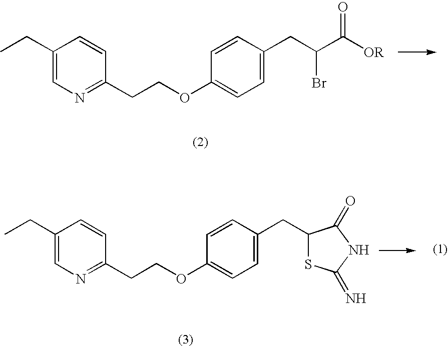

The starting alpha-bromo acid ester (2) is taught to be prepared by Meerwein arylation. This process comprises preparing the corresponding aniline (4), diazotation thereof in the presence of hydrobromic acid, and coupling of the product of diazotation with an acrylic acid ester (5) under catalysis by cuprous oxide as shown below:

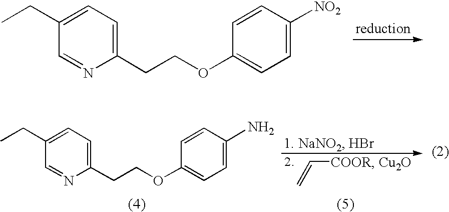

However, forming the alpha-bromo acid ester by the Meerwein arylation reaction can be problematic. The sequence of reactions within this transformation must be controlled precisely. Otherwise the diazo-compound generated during the reaction would react with another nucleophile such as the bromide anion leading to a complicated outcome. Therefore, the reaction often gives a complicated result and lower chemical yield.

Furthermore, the preparation of the starting aniline derivative (4) comprises a hydrogenation step that requires a special apparatus, which gives some difficulties when scaling-up.

EP 0 008 203, which is related to U.S. Pat. Nos. 4,287,200 and 4,481,141, discloses additional glitazones, i.e., not pioglitazone, that can be formed by several possible methods. In addition to the general scheme described in EP 193256, two more synthetic routes are proposed. One technique comprises a cyclization reaction as shown below to form the intended glitazone: 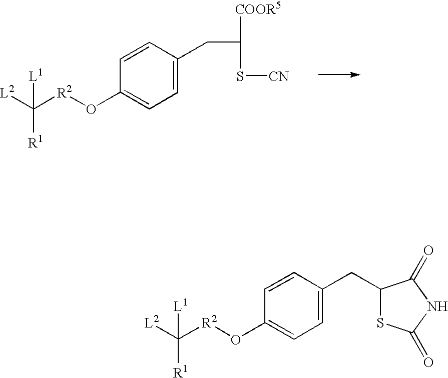
However, the formation of the starting thiocyano compound is not described.

The other technique mentioned in EP 0 008 203 involves coupling the thiazolidine-containing moiety and the substituted alkyl moiety via alkylation of a phenolic oxygen to form the glitazone. If applied to pioglitazone, the reaction would be represented as follows: 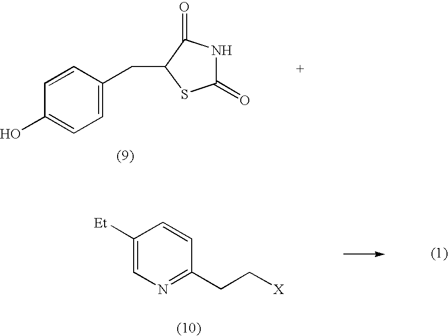
wherein X represents a suitable leaving group. However, reaction conditions for such a pioglitazone-forming alkylation were not explicitly disclosed and furthermore it is believed that the known general reaction conditions of O-alkylation of (9) would provide pioglitazone only in a small yield. Specifically, the low selectivity of the compound (9) for O-alkylation is likely to cause undesired products of side N-alkylation. Also, the compounds of the formula (10) are unstable in that they are susceptible to side elimination reactions upon formation of a vinylpyridine compound of formula (10A), 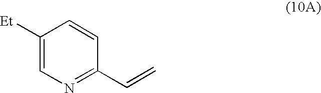
particularly under the conditions that are necessary for nucleophilic substitution reaction with the compound (9). A close ratio of products of N- and O-alkylation of the compound (9) can cause trouble in purification and cause a low chemical yield.

It would be desirable to find alternative processes for making glitazones such as pioglitazone. It would further be desirable to find a process for making glitazones from inexpensive and/or relatively easy to manufacture starting compounds.

### SUMMARY OF THE INVENTION

The present invention is based on the discovery of new processes for making glitazones, especially pioglitazone. Accordingly, a first aspect of the present invention relates to a compound of formula (15): 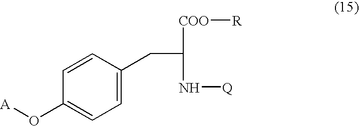
wherein A represents a ring group connected to the oxygen atom by a C₁ to C₆ hydrocarbon chain, R is hydrogen or a C₁-C₄ alkyl, and Q is hydrogen or an amine protecting group, preferably acetyl, trifluoroacetyl, benzoyl, benzyl, or trityl. A preferred compound of formula (15) has the formula (14): 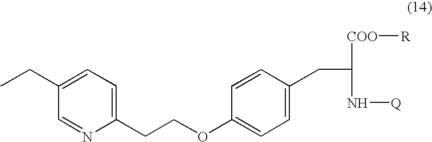
wherein R and Q have the same meaning as in formula (15). These compounds are useful in making glitazones, especially pioglitazone.

Accordingly, another aspect of the present invention relates to a process which comprises converting a compound of formula (15) into a glitazone of formula (16): 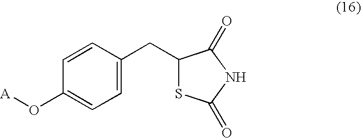
wherein A is as defined above. A preferred process comprises converting a compound of formula (14) into to pioglitazone of formula (1): 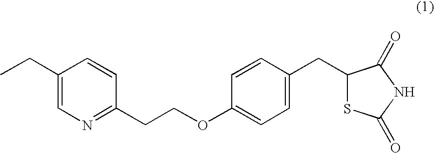
   Preferably Q is hydrogen, or if Q is an amine protecting group, then the conversion step generally includes a deprotection step to provide a free amino-group. Generally the conversion of the compound of formula (14) into pioglitazone comprises forming an intermediate compound of formula (11A), formula (2), or both: 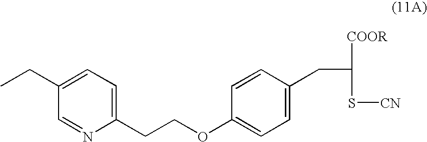
   wherein R represents a hydrogen or a C₁ to C₄ alkyl group; 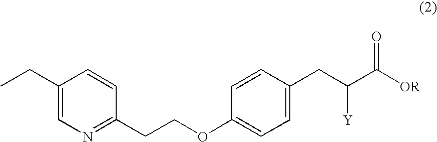
   wherein R represents a hydrogen or a C₁ to C₄ alkyl group and Y represents a leaving group preferably a halogen such as bromo.

In another aspect of the invention, the compounds of formula (14) can be made by reacting a compound of formula (12): 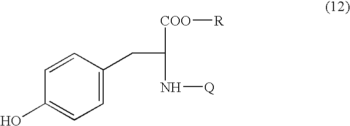
wherein R is hydrogen or a C₁ to C₄ alkyl and Q represents hydrogen or an amine protecting group, with a compound of formula (10): 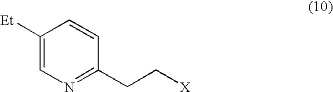
wherein X is a leaving group, to form a compound of formula (14). Such a process can provide the starting compounds of formula (14) via inexpensive starting material, especially tyrosine.

### DESCRIPTION OF THE INVENTION

The present invention relates to the discovery of a novel synthetic route for making glitazones and to novel intermediates useful therein. In general the synthetic route comprises alkylating tyrosine or a protected tyrosine of formula (12) with a suitable alkylating agent to form a compound of formula (15). The amino acid/ester group is then converted to a thiazolidineone ring thereby forming a glitazone (16). The synthesis can be expressed as follows: 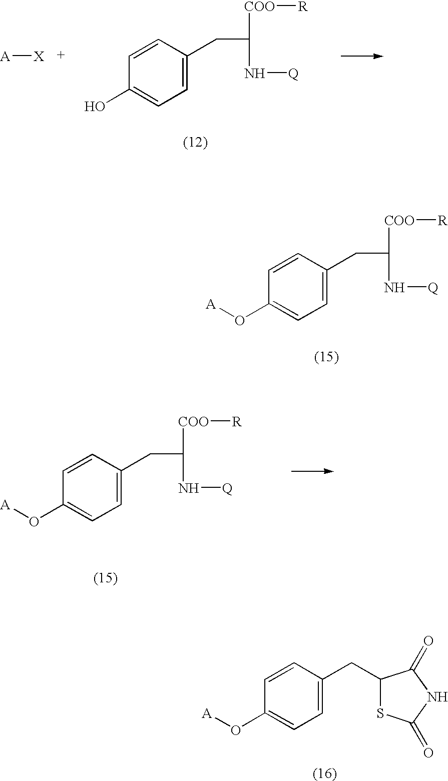
wherein R is hydrogen or a C₁ to C₄ alkyl, Q is hydrogen or an amine protecting group, X is a leaving group and A represents a ring group which, after the alkylation, is connected to the oxygen atom by a C₁ to C₆ hydrocarbon chain. The conversion of compounds of formula (15) is not necessarily performed in a single step. Rather the above scheme is a general approach that can involve multiple reaction steps for each conversion.

It has now been discovered that performing the O-alkylation with tyrosine or a protected derivative thereof provides for higher yields/fewer side products than the O-alkylation of a thiazolidineone as suggested in EP 0 008 203. Further, because tyrosine or a protected derivative thereof is used as the starting material, i.e. a compound of formula (12), the sometimes problematic Meerwein arylation procedure can be avoided. This means that less expensive starting materials can be used in a less expensive process for making glitazones.

The invention is further described with reference to the preferred embodiments wherein pioglitazone is the target glitazone. However, it should be understood that the invention is not limited thereto and these techniques and procedures are equally applicable to other glitazones by selecting the appropriate “A” group.

According to the invention, the compound of formula (11), a sub-genus of formula (12), may be prepared by a process starting from cheap and commercially available tyrosine (6). “Tyrosine” comprises L-tyrosine, D-tyrosine, DL-tyrosine, and mixtures thereof. For instance, the tyrosine may be L-tyrosine. The process is outlined in Scheme 1 below. 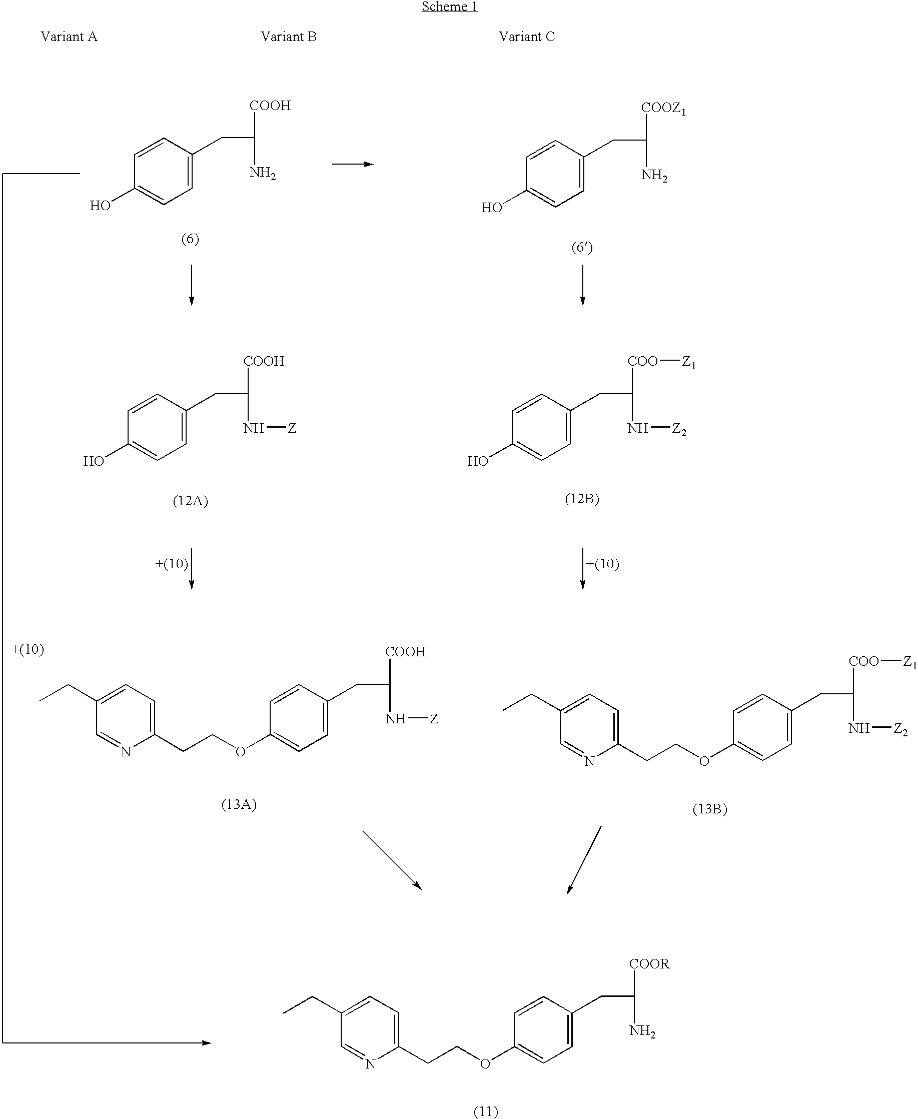
In the above formulas, the variables are as follows:
Z₁ represents a C₁-C₄ alkyl group, including branched chain, and preferably is methyl, ethyl, or isopropyl;
Z and Z₂ independently represent an amine protecting group. Preferred amine protecting groups are acetyl, trifluoroacetyl, benzoyl, benzyl, trityl, benzyloxycarbonyl, formyl, phenacylsulfonyl, and 9-fluorenylmethoxycarbonyl; and
R represents hydrogen or a C₁-C₄ alkyl group, including branched chain, and preferably is methyl, ethyl, or isopropyl.

In the above scheme, compound (10), a sub-genus of the formula “A-X,” is represented by the following formula: 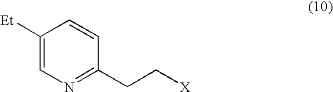
wherein X is a leaving group such as a halogen, methanesulfonyloxy-, or p-toluenesulfonyloxy-group. For clarity, “Et” represents an ethyl group.

The compounds of formulas (6), (12A) and (12B) may be represented by a common general formula (12): 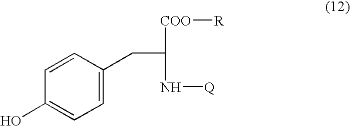
wherein R is as defined above and Q is hydrogen or an amine protecting group, Z.

The compounds of formulas (13A) and (13B) may be represented by a common general formula (13): 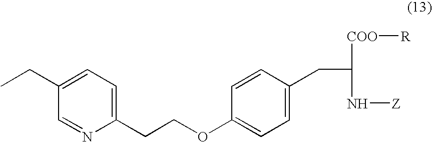
wherein R and Z are as defined above.

The compounds of formulas (13A), 13(B), and (11) may be represented by the following general formula (14): 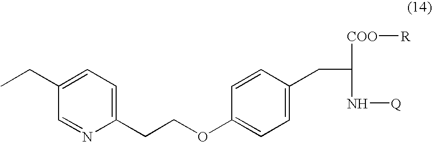
wherein R is as defined above and Q is hydrogen or Z.

Variant A:

This variant comprises direct O-alkylation of tyrosine by the compound (10), wherein X is a suitable leaving group, in a suitable inert solvent in the presence of a suitable base. For example, suitable compounds (10) include 2-ethylpyridin-5ylethyl mesylate or tosylate, i.e., the compound of formula (10) wherein X is methanesulfonyloxy- or p-toluenesulfonyloxy-group, respectively. These compounds may be prepared according to known methods, e.g., by the methods analogous to those shown in EP 0 506 273.

Increased selectivity of the O-alkylation reaction in this variant may be achieved by performing the condensation in a dipolar aprotic solvent, e.g., in dimethylsulfoxide, in the presence of a suitable base (whereby the tyrosine is converted to the corresponding salt with the base) or in the presence of transition metal salts that may form a chelate with carboxy- and amino-groups of tyrosine, for instance nickel or copper salts. It is a disadvantage that tyrosine salt is only moderately soluble in such solvent. Adding water to the solvent increases the solubility but also increases the potential for the undesired N-alkylation. Generally, the maximal suitable content of water in the reaction mixture is about 20%, but the solubility of the sodium salt of L-tyrosine in such a mixture is still less than 4%.

Examples of suitable bases include hydroxides of an alkali metal or an alkaline earth metal, such as sodium hydroxide, potassium hydroxide, calcium hydroxide, and lithium hydroxide. Other suitable bases include quaternary ammonium hydroxides, such as those having at least one bulky substituent such as phenyl, benzyl or aliphatic carbon chain of at least 10 carbons. Such a compound substantially increases the solubility of tyrosine in the dipolar aprotic solvent (thus, less or even no water is necessary) and has a lower potential for catalyzing undesired elimination reactions of the compound (10). An example of a suitable quaternary ammonium hydroxide is benzyltrimethylammonium hydroxide (Triton B). In one embodiment, the tyrosine is dissolved in a methanolic solution of Triton B, the solvent evaporated, and the residue dissolved in dimethylsulfoxide. In this way, it is possible to obtain a concentration in the solution of 20% or higher (w/V) of tyrosine in the solvent. The alkylation reaction can be carried out in the tyrosine solution by adding thereto the compound of formula (10), such as 2-ethylpyridin-5ylethyl mesylate or tosylate, either per se or in the same or a different solvent as the tyrosine solution. Optionally, an additional portion of the same or a different base, such as an alkali metal hydroxide, can be added to the solution. The alkylation reaction generally readily proceeds at ambient temperatures, i.e. 20° C. to 30° C., but elevated temperatures can be used if desired.

Variant B:

In this variant, the conversion process comprises protecting the amino-group of tyrosine with a protective group Z to yield a protected tyrosine of the formula (12A). In a first step, the amino-group of tyrosine is protected against side reactions with alkylating agents by a reaction with a suitable protective agent. The protection may be by an acyl group, such as an acetyl group. Other suitable protective groups Z are benzyl, trityl, benzoyl benzyloxycarbonyl, formyl, phenacylsulfonyl, and 9-fluorenylmethoxycarbonyl group.

Thus, an example of such a protective agent is acetic acid anhydride that produces N-acetylated tyrosine ester, such as N-acetyl tyrosine (compound (12A), Z=acetyl group). The N-acetyl tyrosine may be produced by treating an aqueous suspension of tyrosine with acetic anhydride, evaporation of the solvent, and extraction of the product by acetone. Optionally, the crude product can be re-crystallized, e.g., from 1,4-dioxane or tetrahydrofuran.

The protected, e.g., acetylated, tyrosine is coupled in the next step with the source of 2-ethylpyridin-5ylethyl moiety, i.e., with the compound of formula (10). An example of such a suitable compound is 2-ethylpyridin-5yl ethyl mesylate, the compound of formula (10) wherein X is methanesulfonyloxy-group.

The condensation reaction is advantageously performed by contacting both substrates in a suitable solvent, e.g., in water, a lower alcohol or in a dipolar aprotic solvent such as dimethylformamide, in the presence of a base, e.g., potassium carbonate or an organic amine. Examples of organic amines include those having low nucleophilicity, for instance ethyldiisopropylamine, to suppress undesired elimination reactions of the compound (10). The temperature of the reaction is from ambient to the boiling point of the solvent, such as about 25° C. to 50° C. The course of reaction may be monitored by a suitable method, e.g., by TLC or HPLC.

In a last step, the so obtained intermediate (13A) is deprotected to liberate the amino-group. The choice of deprotection reaction depends on the nature of the protective group as is well known in the art. In the case of N-acetylation, the deprotection may be performed by hydrolysis with an acid, e.g., hydrochloric acid.

Variant C:

In this variant, the conversion process comprises protecting both the carboxy- and amino-groups of tyrosine with suitable protective groups Z₁ and Z₂ to yield a protected tyrosine of formula (12B). In a first step, the tyrosine is converted to an ester (compound (6′), wherein Z₁ is a lower alkyl or benzyl group) by conventional esterification reactions. For example, the esterification may be performed with ethanol and the resulting protected ester is tyrosine ethyl ester (compound (6′), Z₁ is ethyl). Alternatively, the esterification may be performed with isopropanol and the resulting protected ester is tyrosine isopropyl ester (compound (6′), Z₁=isopropyl). Tyrosine esters, particularly tyrosine ethyl ester, are also commercially available. Depending on the mode of preparation, they may be isolated and used in the next step as free bases or acid addition salts (e.g., hydrochlorides). Tyrosine esters are soluble in organic solvents, so that the subsequent reactions may be performed under conditions at which the tyrosine itself does not react.

In a second step, the tyrosine ester reacts with a suitable agent bringing a protective group Z₂ that protects the reactive amino-group. The Z₂ groups for protection of tyrosine esters are essentially the Z-groups as described in the preceding variant. For example, acetylation of the tyrosine ethyl ester or tyrosine isopropyl ester may be performed by reaction with acetic anhydride in a suitable inert solvent, e.g., in a chlorinated hydrocarbon such as dichloromethane, in the presence of a base, e.g., an organic base such as triethylamine.

The protected, e.g., acetylated, tyrosine ester (12B) is coupled in the next step with the source of 2-ethylpyridin-5-ylethyl moiety, i.e., with the compound of formula (10) wherein X is a suitable leaving group. An example of this compound is 2-ethylpyridin-5-ylethyl mesylate as discussed above.

The condensation of the protected tyrosine ester and pyridine compound (10) may be performed by mixing both components in an inert solvent in the presence of a base and allowing them to react at a suitable temperature. The inert solvent may be, e.g., an alcohol (e.g., ethanol), a hydrocarbon (e.g., toluene), and mixtures thereof. The base may be an organic or an inorganic base, e.g., potassium carbonate. The temperature of the reaction is from ambient to the boiling point of the solvent, e.g., from about 25° C. to 110° C. The course of reaction may be monitored by a suitable method, e.g., by TLC or HPLC. It is recommended that the compound (10) is charged in a molar excess, e.g., an excess of about 5 to 50%.

The compound (10) may undergo a side transesterification reaction, by which a side product of the formula (13C) 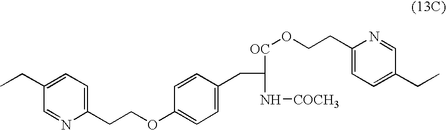
is formed. The side-product may be separated from the desired product (13B) by conventional means, e.g., by chromatography, but this is not necessary. The side product (13C), whenever present in the isolated product (13B), does not harm the next step as it undergoes the same deprotection reaction and yields the same product. The amount of this side product may be reduced by a proper choice of ester group in the tyrosine ester (6′). For instance, isopropyl ester of tyrosine is less susceptible to the transesterification than the tyrosine ethyl ester.

The product of the reaction, i.e., the compound of formula (13B), is deprotected in the last step to liberate free amino group. The deprotection may be total or partial, yielding the compound of formula (11) wherein R is hydrogen or Z₁ group. The means of deprotection depends on the choice of the protective agents. In the case of protective acetylation (Z₂ in compound (13B) is acetyl group), the deprotection is achieved by acidic hydrolysis, e.g., by using hydrochloric acid. Accordingly, the ester group of the compound may also be hydrolyzed during the deprotection, but this is not required because the ester group also reacts during the further conversion to pioglitazone.

By any of the above variants, the desired compound (11) for manufacturing pioglitazone is obtained. Note that compound (11) may be an acid or an ester, depending on the starting material, way of N-protection, and deprotection conditions. Compound (11) may be an acid (R═H), an ester (R═C₁-C₄ alkyl group), or mixtures thereof. Compound (11) may be isolated as a free base or as an acid addition salt with a suitable acid, the later being useful for longer storage or transport. Compound (11) may be purified to the desired degree of purity by known means, e.g., by re-crystallization from a suitable solvent. Alternatively, it may be used in the next step without isolation.

A compound of formula (14), which consists of the compounds of formula (13A), (13B), and (11) can be converted to pioglitazone. The conversion generally involves a cyclization to form the thiazolidinedione ring. Several routes for converting a compound of formula (14) base on forming a compound of formula (11), i.e. if a compound of formula (13A) or (13B) is used, then the amine protecting group is removed as an initial step in the conversion to pioglitazone, are shown below. The invention is not limited thereto and includes any synthetic route whereby a compound of formula (14) is converted to pioglitazone of formula (1).

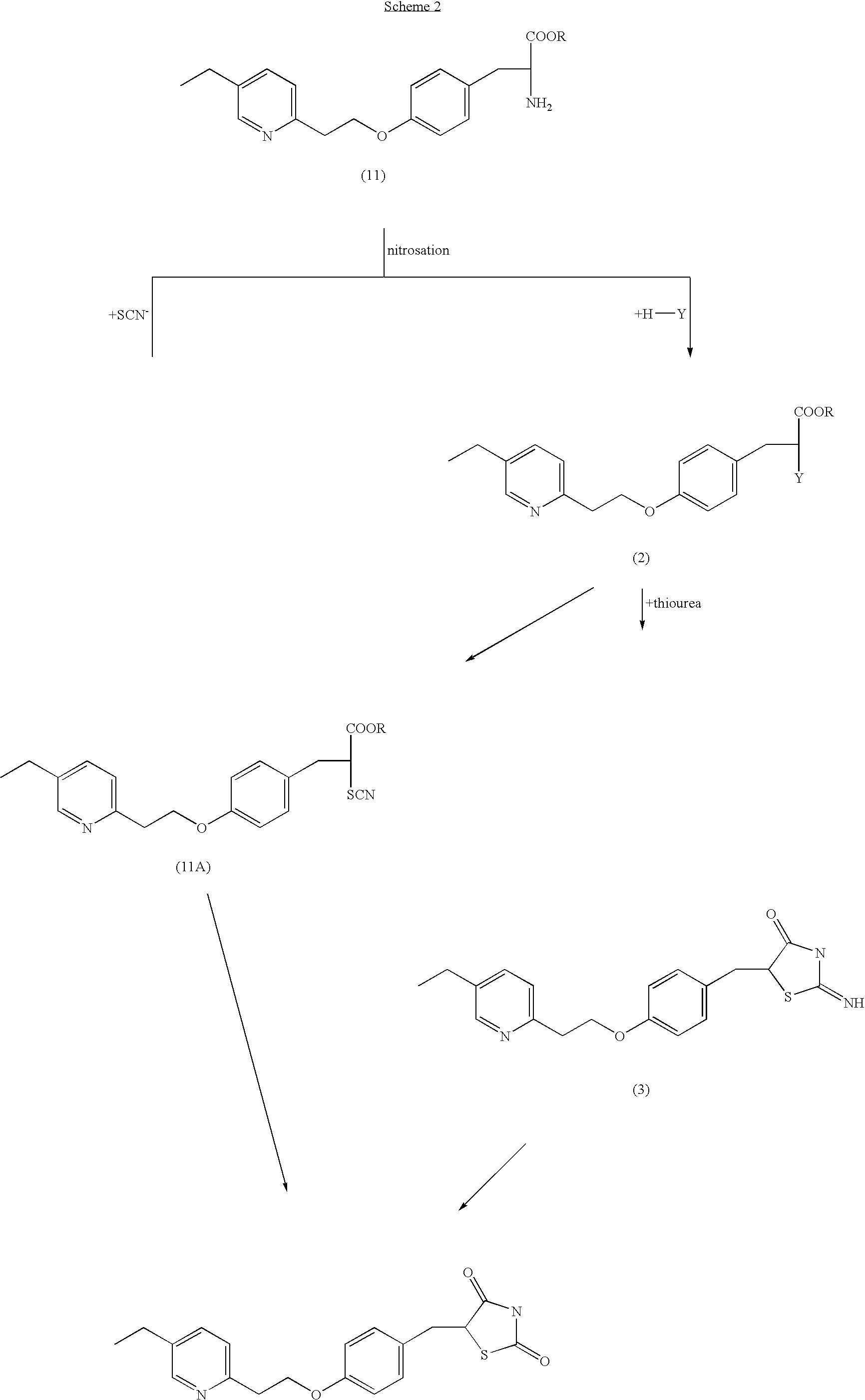

In the first step of Scheme 2, the compound (11) reacts with a nitrosation agent. As used herein a “nitrosation agent” is any compound or combination of compounds that provides a N═O moiety for reaction. Conventional nitrosation agents include nitrous acid, dinitrogen tetroxide, alkyl nitrite (e.g., amylnitrite), or nitrosyl halide (e.g., nitrosyl chloride). Nitrous acid may be generated in situ from a metal nitrite, such as sodium nitrite, and from an acid, such as acetic acid. Also, nitrosyl chloride may be generated in situ, e.g., by a reaction of an alkyl nitrite with a metal halide.

The product of the nitrosation reaction is highly reactive and it may immediately react further without isolation (i.e., in situ). The mechanism of the reaction with the nitrosation agent is not exactly known. While not wishing to be bound by theory, a diazotation reaction is presumed, but the neighboring ester group may also act in the reaction to form an unstable cyclic azo-ester. In any event, the nitrosation product can be converted to various intermediates leading to pioglitazone such as shown in Scheme 2.

For example, conversion can include reaction with an acid H—Y to form a compound of formula (2). Y represents a leaving group while H represents a donatable hydrogen or proton. Examples of H—Y include hydrohalic acid, such as hydrobromic acid, and an alkyl- or aryl-sulfonic acid of the formula R′—SO2-OH, wherein R′ is a lower alkyl (e.g., methyl, ethyl), phenyl, or tolyl group, such as methanesulfonic acid, benzenesulfonic acid, or p-toluenesulfonic acid.

The nitrosation reaction in the presence of an acid H—Y may be performed in a suitable inert solvent, e.g., in water, and at low temperature, such as from −10° C. to 20° C.

The above compounds of formula (2) can be transformed into pioglitazone by any suitable chemical reactions, two of which are shown in Scheme 2. The first route follows the general teaching in EP 0 008 293 and comprises reacting, optionally after isolation from the reaction mixture, the compound of formula (2) with thiourea. The sulfur atom of thiourea replaces the Y-group and the carboxyl group reacts with the amino group of thiourea. As a result, an iminothiazolone ring is formed to obtain the compound of formula (3). The conditions of such reaction are generally known in cases wherein the compound of formula (2) is an ester, i.e., the R group is alkyl. These conditions may also be applied for a compound of formula (2) wherein R is hydrogen (=an acid). In the last step, the imino-thiazolidinone (3) is converted to pioglitazone by a process of hydrolysis that is known in the art as described above.

Alternatively, the compound of formula (2) can be converted to a compound of formula (11A) by reaction with a metal isothiocyanate in an inert solvent. Preferably, the compound of formula (2) is a compound where Y is halogen, especially Br and the metal is an alkali metal, but is not limited thereto.

Once the compound of formula (11a) is formed, it can be cyclized to form pioglitazone by known techniques. For example, the isothiocyanato compound (11A) may be cyclized into the thiazolidine-2,4-dione compound by aqueous hydrolysis, such as in the presence of a catalyst, typically an acid catalyst. Suitable acids include halohydric acids such as hydrochloric acid, sulfuric acid, and alkyl- or aryl-sulfonic acids, such as methane sulfonic acid, ethane sulfonic acid, benzene sulfonic acid, and p-toluene sulfonic acid. The sulfonic acids provide substantially higher yields and purity than the conventional hydrochloric or sulfuric acid as suggested in EP 0 008 203. Methane sulfonic acid, which is a water-containing liquid, may also serve as the solvent for the hydrolysis.

Separately from the formation of a compound of formula (2), the compound of formula (11A) can be formed directly from the nitrosation product by reaction with hydrogen rhodamide. The possibility of conversion of an alpha-amino acid (11) into an alpha rhodano-acid (11A) via nitrosation is a surprising feature. This direct conversion is normally carried out by dissolving the compound (11) in an etheral solvent, e.g., in tetrahydrofuran, in the presence of a proton donor, e.g. an acid such as acetic acid, and with an excess of a metal isothiocyanate especially an alkaline isothiocyanate e.g., lithium isothiocyanate. The treatment of the reaction mixture with a nitrosating agent, especially an alkyl nitrite, e.g., with isoamylnitrite, causes conversion of the compound of formula (11) into (11A). Preferably, the reaction proceeds at ambient or close to ambient temperature, e.g. 15° C. to 30° C. The compound of formula (11A) can then be cyclized by known techniques as described above, to form pioglitazone of formula (1).

The pioglitazone formed by whatever conversion route can be isolated as a base or converted into an acid addition salt, such as a pharmaceutically acceptable acid addition salt. Examples of such salts are pioglitazone hydrochloride, hydrobromide, maleate, fumarate, tartrate, citrate, malate, benzoate, mesylate, and tosylate.

Pioglitazone and its pharmaceutically acceptable salts are valuable pharmaceutical products. It may be used in various pharmaceutical compositions comprising pioglitazone and a pharmaceutically acceptable carrier or diluent. The compositions may be formulated for oral administration. The unit dosage forms include tablets and capsules. The pharmaceutical compositions and final forms comprising pioglitazone may be made by any known process. The tablet compositions may be formulated by known methods of admixture such as blending, filling, and compressing, by means of wet granulation, dry granulation, or direct compression.

Individual unit dose compositions comprising pioglitazone such as tablets or capsules may contain from 1 to 100 mg or 2 to 50 mg of the compound, such as an amount of 2.5, 5, 10, 15, 20, 30, or 45 mg of pioglitazone. Such a composition is normally taken from 1 to 3 times daily, such as once a day. In practice, the physician will determine the actual dosage and administration regimen, which will be the most suitable for the individual patient.

The pioglitazone may be used in the management of various types of hyperglycemia and diabetes, especially Type II diabetes. The present invention also includes the use of pioglitazone of the invention in the manufacture of a medicament for treating and/or preventing any one or more of these disorders. Pioglitazone compositions may be used in medical applications, e.g., in a treatment of certain forms of diabetes, either alone or in combination with other antidiabetic agents, for instance with metformin. The combination may be in a form of a single combination preparation, or by separate administration of drugs containing the above agents.

As mentioned previously, the present invention is not limited to pioglitazone, but can be used to make other glitazones. In this regard, any of the glitazones embraced by EP 0 008 203 or U.S. Pat. No. 6,288,096 can be made by the processes of the present invention; i.e. from tyrosine or a protected tyrosine of formula (12A) or (12B). By replacing the alkylation agent of formula (10) with another suitable reaction partner, generally of the formula A-X, the corresponding analogues of compounds (11) and (13) can be obtained, and then converted to the desired glitazone compound similarly as shown above. For instance, the analogues of compounds (11) and (13) may be represented by formula (15): 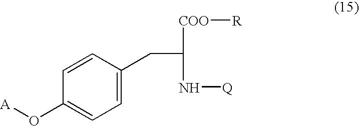
wherein R and Q are as defined above. The compound of formula (15) can be converted into a glitazone of formula (16) via a cyclization route as described above for formula (14); 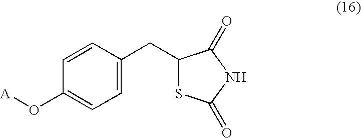
wherein “A” in the above formulas represents a ring group connected to the oxygen atom by a C₁ to C₆ hydrocarbon chain. The ring group is not particularly limited and includes substituted and unsubstituted aromatic and non-aromatic rings, generally having 5 to 12 atoms. Preferably the ring portion of the ring group is a phenyl ring; a 5- or 6-membered heterocyclic ring having one or two heteroatoms selected from nitrogen, oxygen and sulfur atoms, such as a pyridine ring, with remaining ring atoms being carbon atoms; or a bicyclic ring having 8 to 10 atoms wherein up to three atoms can be heteroatoms selected from nitrogen, oxygen and sulfur atoms with the remaining atoms being carbon atoms. The ring portion can be substituted with one or more substituents selected from halogen, C₁ to C₆ alkyl, C₁ to C₆ alkoxy, amino, acyl, sulfonyl, sulfinyl, carboxyl, acylamino, and combinations thereof. The ring portion can be connected to the hydrocarbon chain either directly or via a linking group selected from a carbonyl or amino group. The hydrocarbon chain can be saturated or unsaturated having 1 to 6 carbon atoms. Further, the chain can be interrupted by a linking group as described above and/or can be alkyl substituted with a C₁ to C₄ alkyl group.

Preferred “A” groups include ring groups of the following formulas (a)-(c):

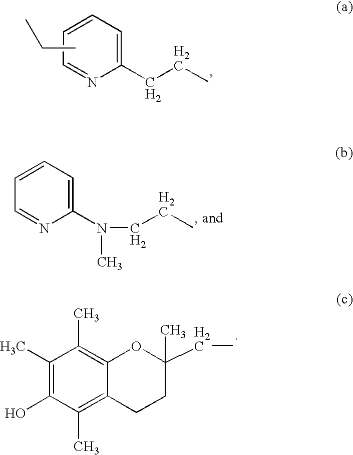

Formula (16) wherein “A” is formula (a) corresponds to pioglitazone and position isomers thereof. Similarly, using formula (b) in formula (16) corresponds to rosiglitazone while using formula (c) is formula (16) corresponds to troglitazone.

The compounds and processes of the present invention allow for the preparation of glitazones, including pioglitazone, from commercially available and cheap tyrosine in acceptable yield and purity.

Each of the patents mentioned above is incorporated herein by reference in its entirety. The present invention will be further illustrated by way of the following Examples. These examples are non-limiting and do not restrict the scope of the invention.

### EXAMPLES

Preparation 1

N-acetyl-L-tyrosine (Compound (12A), Z=acetyl)

18.1 g of L-tyrosine was mixed with 100 mL of water, the mixture was heated to 90-95° C., and 85 mL of acetic anhydride was added dropwise during 2 hours. The light yellow solution was evaporated in a vacuum to give 28.5 g of an oily residue, mixed with 100 mL of acetone, boiled for a few minutes, and the unreacted L-tyrosine was removed by filtration. The filtrate was evaporated in a vacuum, dissolved in 60 mL of 1,4-dioxane. The resulting yellow solution was stirred and seeded. Precipitated crystals were filtered off, air-dried (18.5 g), and recrystallized from tetrahydrofuran.

Preparation 2

N-acetyl Tyrosine Ethyl Ester (Compound (12B), Z₁=ethyl, Z₂=acetyl)

24.6 g of tyrosine ethyl ester hydrochloride was dissolved in 200 ml of dichloromethane. Under cooling (ice-water bath), 20.2 g of triethylamine was added and followed by slow addition of 10.3 g of acetic anhydride. The reaction mixture was further stirred for 1 hour at the same temperature. 200 ml of water was added, and the mixture was stirred for 30 minutes. The resulting layers were separated. In particular, the aqueous layer was extracted with 200 ml of dichloromethane. The organic layers were combined and dried over sodium sulfate and concentrated in a vacuum to give 29.3 g of an oily product.

Preparation 3

2-(5-ethyl-pyridin-2-yl)-ethyl Methanesulfonate

30.2 g of 2-(5-ethylpyridin-2-yl)ethanol was dissolved in 300 ml of toluene. Under cooling in an ice water bath, 20.2 g of triethylamine was added followed by slow addition of 22.9 g methane sulfonylchloride. After completion of the addition (30 minutes), the reaction mixture was stirred for 1 hour at approx. 3° C. The reaction mixture was washed with 2×100 ml of water, 50 ml of brine, and dried over sodium sulfate.

The obtained toluene solution was used for subsequent synthesis.

In some cases, as discussed below, 100 ml of the solution was evaporated to obtain an oily product (14.02 g).

Preparation 4

L-tyrosine Isopropylester

60 g of L-tyrosine was suspended in 420 mL of isopropanol, gaseous hydrogen chloride was introduced, the temperature was slowly raised, and the mixture was heated under reflux for 8 hours. The reaction mixture was partially evaporated under diminished pressure, and the concentrated solution was poured into a mixture of 1200 mL of 5% sodium hydrogen carbonate and 95% dichloromethane. The dichloromethane layer was twice extracted with 60 ml of water, dried with magnesium sulfate, and the drying agent was removed by vacuum. The filtrate was concentrated in a vacuum. Precipitated crystals were collected by vacuum. The cake was air-dried to give 48 g of a first batch with a m.p. of 121-124° C. A second batch of 11.8 g with a m.p. 118-123° C. was obtained from the filtrate. The yield of L-tyrosine isopropylester was 78%.

Preparation 5

N-acetyl-L-tyrosine Isopropyl Ester

2.0 g of isopropyl-L-tyrosine was suspended in 2 mL of acetic acid and 9 mL of acetic anhydride was added dropwise. The resulting solid was dissolved, and the mixture was heated at 90° C. for 6 hours. The reaction mixture was cooled, diluted with 10 mL of water, and neutralized with 0.5 g of sodium hydrogencarbonate. The mixture was twice extracted with 10 mL of dichloromethane. The dichloromethane extracts were combined and washed with sodium hydroxide solution, water, and evaporated in a vacuum. The resulting oil was stirred with diethylether. The resulting crystals were filtered off and air-dried to give 1.3 g of a product with a m.p. of 90-92° C. The yield was 54%. The structure of the product was confirmed by NMR.

### Example 1

### Preparation of 2-amino-3-{4-[2-(5-ethyl-pyridin-2-yl)-ethoxy]-phenyl}-propionic Acid (Compound (11) with R═H) by Alkylation of L-tyrosine Sodium Salt in DMSO

10 g of L-tyrosine was dissolved in 43 mL of 1 M NaOH, 245 mL of dimethylsulfoxide was added, followed by addition of 14 g of 2-(5-ethyl-pyridin-2-yl)-ethyl methanesulfonate (Preparation 3). The reaction mixture was stirred over 72 hours, solvents were removed in a vacuum, and the residue was dissolved in 100 mL of water. The water solution was neutralized with 6N hydrochloric acid. A precipitate was filtered off and washed with water. Recrystallization from hot methanol-water solution yielded 3.1 g of 2-amino-3-{4-[2-(5-ethyl-pyridin-2-yl)-ethoxy]-phenyl}-propionic acid (R═H).

### Example 2

### Preparation of 2-amino-3-{4-[2-(5-ethyl-pyridin-2-yl)-ethoxy]-phenyl}-propionic Acid (Compound (11) with R═H) by Alkylation of L-tyrosine Lithium Salt in DMSO

14 g of L-tyrosine was dissolved in a mixture of 12 g of lithium hydroxide and 120 mL of water. Then, 400 mL of dimethylsulfoxide was added, followed by addition of 23.5 g of 2-(5-ethyl-pyridin-2-yl)-ethyl methanesulfonate (Preparation 3) in 100 mL of toluene. The reaction mixture was stirred for 48 hours at ambient temperature and then five times extracted with 20 mL of toluene. The pH of the dimethylsulfoxide layer was adjusted to 8 with 6N aqueous hydrochloric acid (1:1). The mixture was stirred, the precipitate was filtered off (8.9 g of lithium salt of L-tyrosine), and the cake was washed with hot ethanol. The ethanol was evaporated, and the residue added to the filtrate. The filtrate was acidified with hydrochloric acid to pH 2, and solvent was removed at 50° C. in a vacuum. The residue was dissolved in water and neutralized with a 25% aqueous solution of sodium hydroxide. The precipitate was removed by filtration to give 7.0 g of a solid. The yield was 22%.

### Example 3

### Preparation of 2-amino-3-{4-[2-(5-ethyl-pyridin-2-yl)-ethoxy]-phenyl}-propionic Acid (Compound (11) with R═H) by Alkylation of L-tyrosine benzyltrimethylammonium Salt in DMSO

10 g of L-tyrosine was mixed with 25 mL of 40% benzyltrimethylammonium hydroxide in methanol. The mixture was heated and methanol was removed in a vacuum. Then, 50 mL of dimethylsulfoxide was added, and the mixture was heated until all solids were dissolved (90° C.). The solution was left to cool, and 3.0 g of sodium hydride was added. Then, a solution of 2.3 g of 2-(5-ethyl-pyridin-2-yl)-ethyl methanesulfonate (Preparation 3) in dimethylsulfoxide and 2.5 g of solid sodium hydride were added during 6 hours in portions. The reaction mixture was left to stir overnight at ambient temperature. Then, 150 mL of acetone was added, and the precipitate was filtered off and dissolved in 50 mL of water and acidified with hydrochloric acid. The solution was extracted with ethylacetate, and the water layer was neutralized with sodium hydroxide solution. The precipitate was filtered off, mixed with 200 mL of ethanol, acidified with 8 mL of concentrated hydrochloric acid, and heated under reflux for 30 minutes. Undissolved solids were removed by filtration. The filtrate was concentrated in a vacuum and cooled. Precipitated crystals were collected by filtration and air-dried to give 10.5 g of 2-amino-3-{4-[2-(5-ethyl-pyridin-2-yl)-ethoxy]-phenyl}-propionic acid hydrochloride.

### Example 4

### Preparation of 2-amino-3-{4-[2-(5-ethyl-pyridin-2-yl)-ethoxy]-phenyl}-propionic Acid (Compound (11) with R═H) by Alkylation of L-tyrosine Chelate

10 g of L-tyrosine was dissolved in 54 mL of 2 M NaOH, and 30 mL of a solution of 6.8 g of copper sulfate in water was added. The mixture was heated to 60° C. for 10 minutes, and 88 mL of a toluene solution of 12.6 g of 2-(5-ethyl-pyridin-2-yl)-ethyl methanesulfonate (Preparation 3) was added. The mixture was heated and maintained at 50° C. for 5 hours. The reaction mixture was diluted with water, extracted twice with 20 mL of ethylacetate, and 2 g of sodium sulfide was added to the water layer. A brown precipitate was filtered off, and the filtrate was acidified with 6N hydrochloric acid. Water was removed in a vacuum and the residue was mixed with hot methanol-water solution. Undissolved crystals were removed by filtration, and the product crystallized from filtrate on cooling. 1.5 g of 2-amino-3-{4-[2-(5-ethyl-pyridin-2-yl)-ethoxy]-phenyl}-propionic acid (compound (11) with R═H) was obtained.

### Example 5

### 2-amino-3-{4-[2-(5-ethyl-pyridin-2-yl)-ethoxy]-phenyl}-propionic Acid

### A. Preparation of 2-acetylamino-3-{4-[2-(5-ethyl-pyridin-2-yl)-ethoxy]-phenyl}-propionic Acid (Compound (13A) with Z=acetyl)

2.5 g of N-acetyl-L-tyrosine was dissolved in 20 mL of isopropanol, and 4 mL of N-ethyldiisopropylamine was added. Then, a solution of 2.1 g of 2-(5-ethyl-pyridin-2-yl)-ethyl methanesulfonate in 16 mL of toluene was added, and the reaction mixture was heated under reflux for 13 hours. Isopropanol was partially removed in a vacuum, and the residue was diluted with 50 mL of water and twice extracted with 5 mL of toluene. The water layer was neutralized with 6N hydrochloric acid and twice extracted with dichloromethane. The dichloromethane extracts were combined and dried with sodium sulfate, and then the solvent was evaporated in a vacuum to give 2.0 g of oily product. The yield was 69%.

### B. Deacetylation of Alkylated Product of Example 5A with Hydrochloric Acid

1.7 g of the oily product of Example 5A was heated under reflux with 50 mL of 10% HCl for 3 hours. The reaction mixture was concentrated in a vacuum to an oil that was dissolved in 10 mL of water, and ammonia was added to adjust the pH to 7.0. Precipitated crystals were filtered off and air-dried to give 1.2 g of an intermediate with a m.p. of 212-217° C.

### Example 6

### Preparation of 2-acetylamino-3-{4-[2-(5-ethyl-pyridin-2-yl)-ethoxy]-phenyl}-propionic Acid (Compound (13A) with Z=acetyl) Hydrochloride

2.5 g of N-acetyl-L-tyrosine (Preparation 1) was dissolved in a solution of 1.0 g of potassium carbonate and 1.2 mL of water. A solution of 2.35 g of 2-(5-ethyl-pyridin-2-yl)-ethyl methanesulfonate (Preparation 3) dissolved in 16 mL of toluene was added, and the mixture was heated at 50° C. for 3 hours. Then, the reaction mixture was twice extracted with ethylacetate. The water phase was acidified with diluted hydrochloric acid and evaporated in a vacuum. The residue was heated with 30 mL of hot isopropanol. Solids were removed by filtration and the filtrate cooled. Precipitated crystals were filtered off to give 1.8 g of 2-acetylamino-3-{4-[2-(5-ethyl-pyridin-2-yl)-ethoxy]-phenyl}-propionic acid.

### Example 7

### 2-amino-3-{4-[2-(5-ethyl-pyridin-2-yl)-ethoxy]-phenyl}-propionic Acid

### A. Preparation of Ethyl 2-acetylamino-3-{4-[2-(5-ethyl-pyridin-2-yl)-ethoxy]-phenyl}-propionate

29.34 g of N-acetyl tyrosine ethylester (Preparation 2), 200 ml of ethanol, 14.0 g of potassium carbonate, and 200 ml of a toluene solution of 2-(5-ethyl-pyridin-2-yl)-ethyl methanesulfonate (Preparation 3) were refluxed under stirring for 3 hours. Then, 14.0 g of potassium carbonate and 50 ml of the toluene solution of 2-(5-ethyl-pyridin-2-yl)-ethyl methanesulfonate were added, and the reflux was continued for another 4 hours. The mixture was cooled in a water bath, and 100 ml of water was added under cooling. The mixture was concentrated under reduced pressure to approx. 50% volume and the remaining solution was extracted with 2×200 ml of ethyl acetate. The organic layer was concentrated to give an oily product, which was used in the next step directly.

### B. Deprotection to 2-amino-3-{4-[2-(5-ethyl-pyridin-2-yl)-ethoxy]-phenyl}-propionic Acid

The crude material from Example 7A was mixed with 370 ml of 10% HCl and stirred at approx. 100° C. for 4 hours. The mixture was concentrated under reduced pressure (approx. 50 ml was removed), and the concentrate was neutralized to a pH of approx. 7.0 by adding 15% ammonium hydroxide. The separated solid was collected by filtration and washed with 2×50 ml of water. After drying, 25 g of crude product was obtained.

### Example 8

### 2-amino-3-{4-[2-(5-ethyl-pyridin-2-yl)-ethoxy]-phenyl}-propionic Acid

### A. Preparation of isopropyl 2-acetylamino-3-{4-[2-(5-ethyl-pyridin-2-yl)-ethoxy]-phenyl}-propionate

5.0 g of isopropyl N-acetyl-L-tyrosine was dissolved in 25 mL of isopropanol, and 3.5 g of potassium carbonate were added. Then, 2.5 g of 2-(5-ethyl-pyridin-2-yl)-ethyl methanesulfonate (Preparation 3) dissolved in 17 mL of toluene was added, and the reaction mixture was heated under reflux for 23 hours. Then, another portion (2.5 g) of Preparation 3 dissolved in 17 mL of toluene was added, and the reaction mixture was heated for 23 hours. The solution was evaporated in vacuum. The residue was twice extracted with 20 mL of toluene, and the toluene extract was evaporated in a vacuum to give 5.21 g of oily product. The oily product was dissolved in 10 mL of diethylether and crystallized on stirring. The resulting solid was filtered and air-dried to give 4.5 g of a product with a m.p. of 73-81° C. The yield was 58%.

### B. Deprotection to 2-amino-3-{4-[2-(5-ethyl-pyridin-2-yl)-ethoxy]-phenyl}-propionic Acid

1.0 g of the solid product from Example 8A was heated under reflux with 50 mL of 10% HCl for 4 hours. The reaction mixture was concentrated in a vacuum to give 1.2 g of oil that was dissolved in 50 mL of water. Ammonia was added to adjust the pH to 7.0. Precipitated crystals were filtered off and air-dried to give 0.40 g of an intermediate with a m.p. of 214-221° C.

### Example 9

### Conversion of 2-amino-3-{4-[2-(5-ethyl-pyridin-2-yl)-ethoxy]-phenyl}-propionic Acid to Pioglitazone

### A. 2-bromo-3-{4-[2-(5-ethyl-pyridin-2-yl)-ethoxy]-phenyl}-propionic Acid

2.0 g of 2-amino-3-{4-[2-(5-ethyl-pyridin-2-yl)-ethoxy]-phenyl}propionic acid was suspended in 30 ml of water. 3.2 g of 47% aqueous HBr was added to the suspension under stirring. Under cooling (ice-water bath), a solution of 1.4 g of sodium nitrite in 20 ml of water was added within 3 hours. During the addition, 20 ml of acetone was added in portions to dissolve a sticky solid that was generated. After further stirring for 1 hour at approx. 3° C., the mixture was concentrated to remove acetone. The concentrate was extracted with 3×50 ml of ethyl acetate. The organic layers were combined and dried over sodium sulfate and evaporated to give a crude product (2.14 g).

### B. Pioglitazone Hydrochloride

2.14 g of the crude product from Example 9A was dissolved in 50 ml of ethanol. 760 mg of thiourea and 820 mg of sodium acetate were added. The solution was refluxed for 3 hours and concentrated to remove most of the ethanol.

20 ml of 3N HCl was added to the residue, and the mixture was refluxed for 18 hours. After cooling down to room temperature, the mixture was neutralized by 28% aqueous ammonia. The generated solid was collected by filtration and washed with 2×10 ml of ethanol. This yielded 1.12 g of greyish solid.

### Example 10

### Conversion of 2-amino-3-{4-[2-(5-ethyl-pyridin-2-yl)-ethoxy]-phenyl}-propionic acid into 2-thiocyanato-3-{4-[2-(5-ethyl-pyridin-2-yl)-ethoxy]-phenyl}-propionic Acid

2.8 g of 2-amino-3-{4-[2-(5-ethyl-pyridin-2-yl)-ethoxy]-phenyl}-propionic acid was mixed with 30 mL of tetrahydrofuran, 5 mL of acetic acid, and 2.4 g of lithium thiocyanate. Then, 2.4 mL of isopentylnitrite was added in portions during 4 hours. The reaction mixture was stirred overnight. The resulting solution was evaporated in a vacuum, and the oily residue was heated under reflux with 40 mL of ethylacetate. The resulting suspension was left to cool. Crystals were separated by filtration to give 1.5 g of product with a m.p. of 175-178° C. The identity was confirmed by NMR and IR spectra.

### Example 11

### Pioglitazone (from 2-thiocyanato-3-{4-[2-(5-ethyl-pyridin-2-yl)-ethoxy]-phenyl}-propionic Acid)

0.50 g of 2-thiocyanato-3-{4-[2-(5-ethyl-pyridin-2-yl)-ethoxy]-phenyl}-propionic acid was dissolved in 10 mL of methanesulfonic acid, and the resulting brown solution was stirred overnight. Then, the reaction mixture was poured onto crushed ice upon stirring and sodium hydrogencarbonate was added in portions to neutralize the mixture. Precipitated light brown crystals were filtered off and dried to give 0.70 g of raw product, which was further purified by mixing with 12% ethanolic solution of hydrogen chloride. The undissolved portion was filtered off, and the filtrate was precipitated with sodium bicarbonate, which gave crystals of pioglitazone with a m.p. of 180-184° C.

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

1. A compound of formula (15) 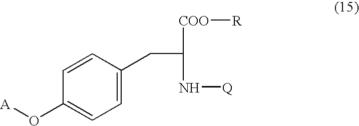
wherein A represents a ring group connected to the oxygen atom by a C₁ to C₆ hydrocarbon chain, R is hydrogen or a C₁-C₄ alkyl, and Q is hydrogen or an amine protecting group.

2. The compound according to claim 1, wherein A represents one of the following formulas (a)-(c):

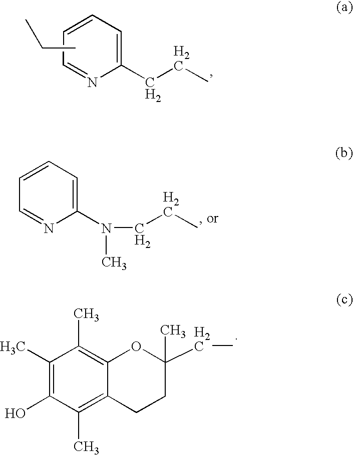

3. The compound of according to claim 1, having the formula (14): 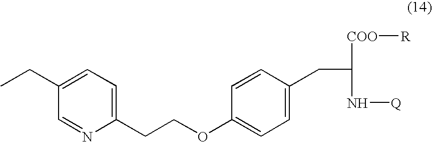
wherein R is hydrogen or a C₁-C₄ alkyl, and Q is hydrogen or an amine protecting group.

4. The compound according to claim 3, wherein said amine protecting group is selected from the group consisting of acetyl, trifluoroacetyl, benzoyl, benzyl, or trityl.

5. The compound according to claim 3, wherein Q is hydrogen.

6. The compound according to claim 5, wherein R is hydrogen.

7. The compound according to claim 3, wherein Q is acetyl, trifluoroacetyl, benzoyl, benzyl, or trityl.

8. The compound according to claim 7, wherein R is a C₁-C₄ alkyl.

9. The compound according to claim 8, wherein R is ethyl.

10. The compound according to claim 9, wherein Q is acetyl.

11. The compound according to claim 6, wherein Q is acetyl.

12. The compound according to claim 3, wherein R is hydrogen.

13. The compound according to claim 3, wherein R is ethyl.

14. A process which comprises converting a compound of formula (15) 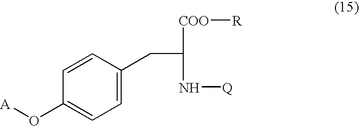
wherein A represents a ring group connected to the oxygen atom by a C₁ to C₆ hydrocarbon chain, R is hydrogen or a C₁-C₄ alkyl, and Q is hydrogen or an amine protecting group, to form a glitazone of formula (16): 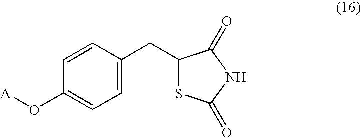
wherein A is as defined above.

15. The process according to claim 14, wherein A represents one of the following formulas (a)-(c):

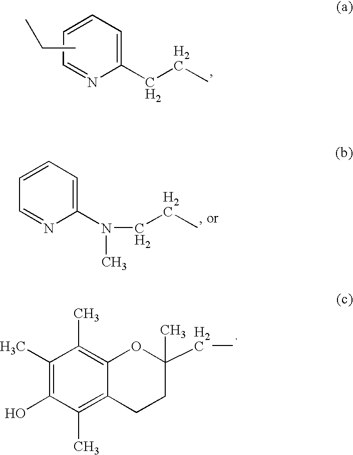

16. The process according to claim 15, which comprises:
converting a compound of formula (14): 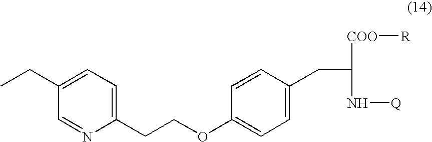
wherein R is hydrogen or a C₁-C₄ alkyl, and Q is hydrogen or an amine protecting group, into a pioglitazone of formula (1):

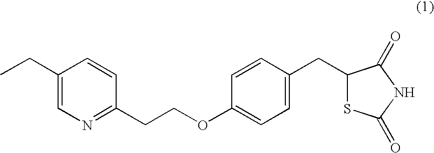

17. The process according to claim 16, wherein Q is hydrogen.

18. The process according to claim 17, wherein R is hydrogen.

19. The process according to claim 17, wherein R is a C₁ to C₄ alkyl.

20. The process according to claim 19, wherein R is ethyl.

21. The process according to claim 17, wherein said converting comprises forming an intermediate of formula (11A) 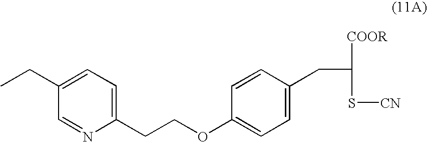
wherein R is a C₁ to C₄ alkyl; and
cyclizing said compound of formula (11A) to form said pioglitazone of formula (1).

22. The process according to claim 21, wherein said cyclizing comprises aqueous hydrolysis of the compound of formula (11A).

23. The process according to claim 22, wherein said cyclizing is catalyzed by an alkyl- or aryl-sulfonic acid.

24. The process according to claim 17, wherein said converting comprises forming an intermediate of formula (2): 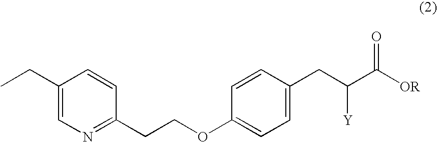
wherein Y represents a leaving group and R is hydrogen or a C₁ to C₄ alkyl, and transforming said compound of formula (2) into said pioglitazone of formula (1).

25. The process according to claim 24, wherein said transforming of said compound of formula (2) into said pioglitazone of formula (1) comprises:
reacting said compound of formula (2) with thiourea to form a compound of formula (3): 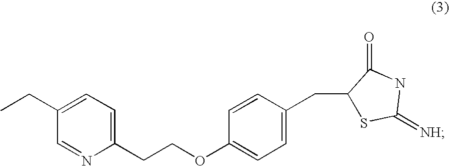
and
hydrolyzing said compound of formula (3) to form said pioglitazone of formula (1).

26. The process according to claim 24, wherein R in formula (2) is a C₁ to C₄ alkyl.

27. The process according to claims 26, wherein Y is a halogen.

28. The process according to claim 16, which further comprises reacting a compound of formula (10) with a compound of formula (12) to form said compound of formula (14), wherein formula (10) is: 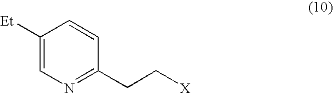
wherein X is a leaving group; and formula (12) is: 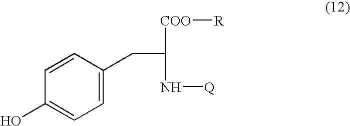
wherein R is hydrogen or a C₁ to C₄ alkyl and Q is hydrogen or an amine protecting group.

29. The process according to claim 28, wherein Q is an amine protecting group in said compounds of formula (12) and (14).

30. The process according to claim 29, wherein said converting of said compound of formula (14) into pioglitazone includes deprotecting said amine protecting group in said compound of formula (14).
